(19) United States
(12) Patent Application Publication
(10) Pub. No.: US 2015/0030368 A1
(43) Pub. Date: Jan. 29, 2015

(54) Single Use Deodorant

(71) Applicants: Michael W. Connolly, Keller, TX (US); Lee P. Sander, McKinney, TX (US)

(72) Inventors: Michael W. Connolly, Keller, TX (US); Lee P. Sander, McKinney, TX (US)

(21) Appl. No.: 13/975,315

(22) Filed: Aug. 24, 2013

Related U.S. Application Data
(60) Provisional application No. 61/958,278, filed on Jul. 24, 2013.

(51) Int. Cl. A61K 8/02; A61Q 19/00; A45D 40/00; A61Q 15/00

(52) U.S. Cl. CPC A61K 8/0204; A61Q 15/00; A61Q 19/00; A45D 40/00; A45D 2040/0012
USPC 401/49

(57) ABSTRACT

A single use, disposable ‘card’ for transferring a deodorant or other cosmetic product to the human body. The card can be discreetly carried and used, followed by easy disposal of the card and packaging. The card is coated (on one or both sides) with a deodorant product, and is roughly the size of a credit card, with variations in height and width as required. A removable protective covering is provided over the area of the card containing the deodorant product, or is large enough to contain the entire card.

### CROSS REFERENCE TO RELATED APPLICATIONS

This application references and claims the benefit of the content and priority date of the provisional application #61958278 which was filed Jul. 24, 2013.

### BACKGROUND OF THE INVENTION

This invention relates to an improvement in the convenience and application of personal care products. Specifically, in its preferred embodiment, this invention presents a disposable card style applicator as a base, that is coated with (on one or both sides) and acts as a carrier for, a deodorant and/or antiperspirant compound or composition (product).

Deodorants and antiperspirants are substances applied to the body to reduce body odor and underarm moisture. Certain products can also be used on other areas of the body that produce sweat and odor, such as the feet.

The first commercial offering for a deodorant product was developed in the late 1800's. Through invention and product development, the active ingredients as well as the method of application have evolved from the original paste sold in the late 1800's. Methods of application appeared that included roll-on, sticks, gels and aerosols. Not unexpectedly, these improved products became popular because of improvements to the ease of application and the aesthetics of the resulting body area following application/use.

Development efforts of the Bristol-Myers company introduced a new style of applicator in the 1950's, known then by the name Ban Roll-On. Later, the aerosol delivery method became very popular in the early 1960's and shortly accounted for half of all antiperspirants sold in the US. However, in the late 1970's the aerosol style product decreased in popularity because of safety concerns regarding both the active ingredients of the product as well as the propellants used in the aerosol. Thus, products that used what is known as the ‘stick’ style of product became the dominate/preferred product, and remain so today.

Contributing to the need for improvements in the convenience and application of personal care products is the fact that through the past few generations, our society has changed—with people squeezing more things into their day, having less free time but more they want to accomplish during that free time, more discretionary income, and a greater independence at younger ages. As a people we are also living longer, healthier lives and are able to be active for more years.

Additionally it's no longer just a small minority of the populace that has the time and money to enjoy this type of life. Rather, it's available to a much wider spectrum of people.

The result has been a steady increase in the mobility of our population, busier days with tighter time schedules, and a wider variety of strenuous activities that we participate in. For example, business trips are common in the 21^{st} century, and may span many days and nights. This requires, per our accepted social standards, that the person on the trip bathes while on this trip, and subsequently applies a deodorant product. It may also mean that delays in travel, for example, dictate that a ‘freshening up’ is required immediately without the time or convenience of a place to do so.

Another example of mobility is the increased frequency of individuals, couples, small groups and families going camping or “getting off the grid” to wilderness and undeveloped areas, many of which may not be provisioned with typical bathing facilities.

We also are a society that is increasingly aware of and conscience of our health, especially with the ever increasing expected live spans and the slowing of deterioration in our health, fitness and ability to live an active life. Thus we frequently, and for more years of our lives, participate in exercise classes, working out in the gym, running, biking and playing team sports. Our evolving culture and improving health conditions allow many sports to be enjoyed and engaged in, sometimes at any time during our day or night. Many executives will take in a game of squash during lunchtime, or play a round of golf or tennis with business partners, coworkers, and existing or potential customers. It is considered a standard part of doing business.

In each of these scenarios, as well as many others, we want to bathe or ‘freshen up’ wherever we are, without the need to return home to do so. This process includes application of a deodorant product, which we must carry along with us to wherever we are, and which must be readily at hand, quickly used, and easily/discreetly disposed of.

None of the prior art discloses or even addresses an increasingly frequent issue encountered as a combination of these evolutions in our society (increased mobility; tighter schedules; more people engaged in strenuous ‘sweat producing’ activities; and the need to discreetly carry, use and dispose of a deodorant product). Prior art and the products available today are typically large and bulky enough that they are not easily transported with us while we are living our active lives. They also contain a quantity of deodorant product sufficient for more than a single use, typically it may be for weeks of use, so is not economical to dispose of following a single use.

What is required is a small, convenient deodorant containing product that is discreetly carried with us, is simple to apply, can be used anywhere and anytime, and can be disposed of easily when used up.

### BRIEF SUMMARY OF THE INVENTION

This invention relates to an improvement in the convenience and application of personal care products. The object of the present invention is to enable an individual to discreetly carry and easily use a deodorant or other personal care or cosmetic product, followed by easily disposing of the applicator and packaging. This invention consists of a single use, disposable ‘card’ for transferring a deodorant or other cosmetic product to the human body.

Specifically, in its preferred embodiment, this invention presents a card style applicator that is coated (on one or both sides) with a deodorant product. This card can be roughly the size of a credit card, with variations in height and width as required. The personal care or cosmetic product may be applied over all or a substantial portion of the card surface, in any pattern (rectangular, a series of dots, a series of lines or ribs, etc.), with the potential given to a space left for the user to grasp during use.

A protective covering is provided over at least the area of the card containing the deodorant product. This cover can also be a sealed bag that contains the entire card.

### BRIEF DESCRIPTION OF THE DRAWINGS

FIG. 1 shows an example of the preferred embodiment of the invention, with a provided space A to grasp the card, and a roughly rectangular area B covered with the deodorant product itself.

FIG. 2 shows an example of the preferred embodiment of the invention, with a cover C over the area of deodorant product.

FIG. 3 shows an example of the preferred embodiment of the invention, with a ‘bend and break’ scoring D used to remove the cover.

FIG. 4 shows an example of the preferred embodiment of the invention, with a tear strip E incorporated to remove the cover.

FIG. 5 shows an example of the preferred embodiment of the invention, with a peel back adhesive area F around the perimeter of the cover and tab G incorporated to remove the cover.

FIG. 6 shows an example of the preferred embodiment of the invention, with a solid roughly rectangular area H of deodorant product applied.

FIG. 7 shows an example of the preferred embodiment of the invention, with an area J of spaced apart dots of deodorant product applied.

FIG. 8 shows an example of the preferred embodiment of the invention, with an area K covered by a series of ribs of deodorant product applied.

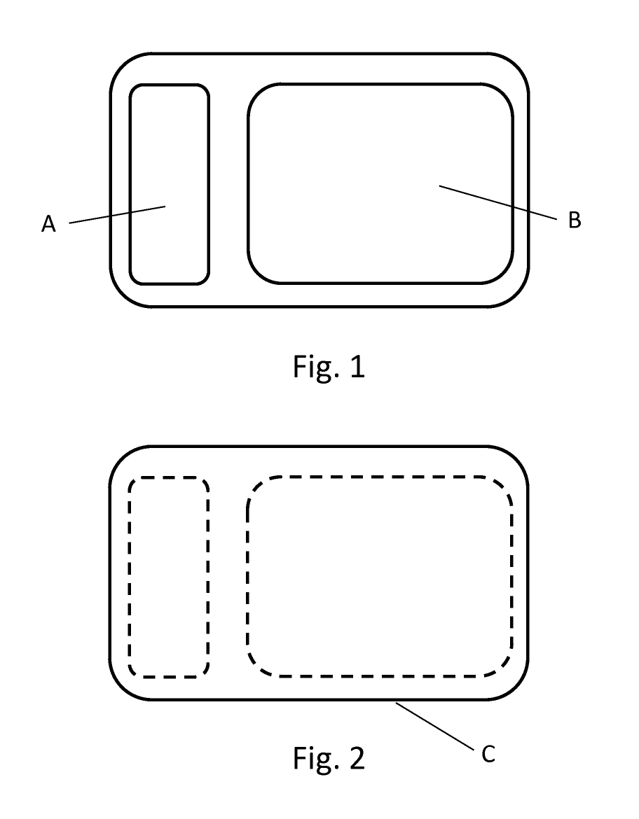

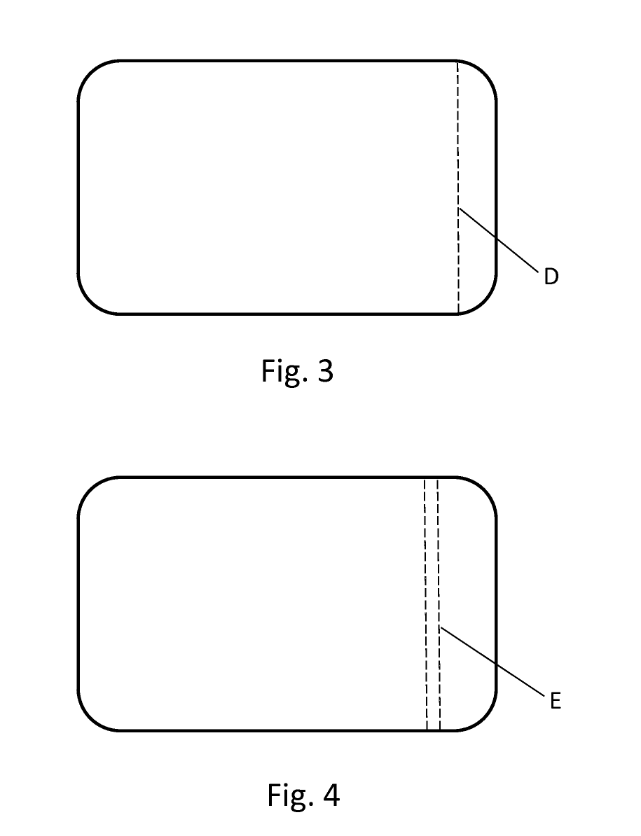

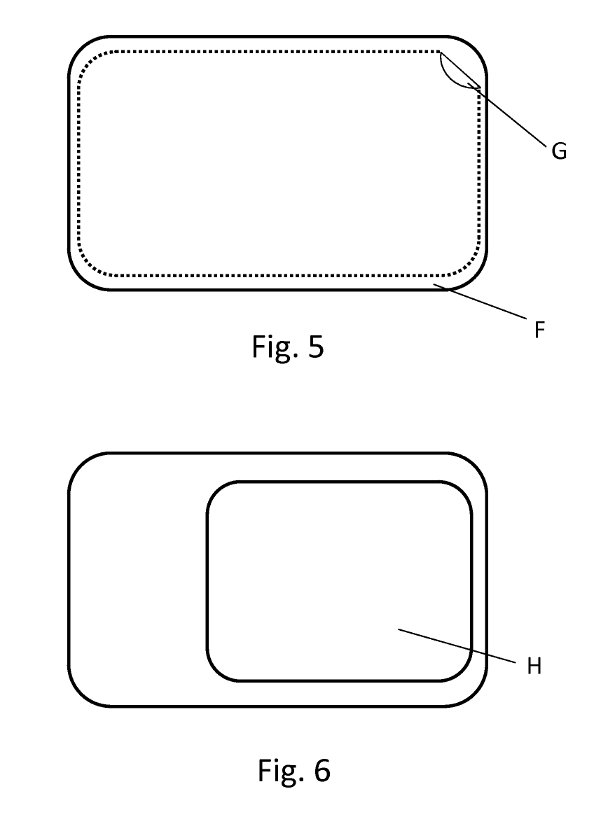

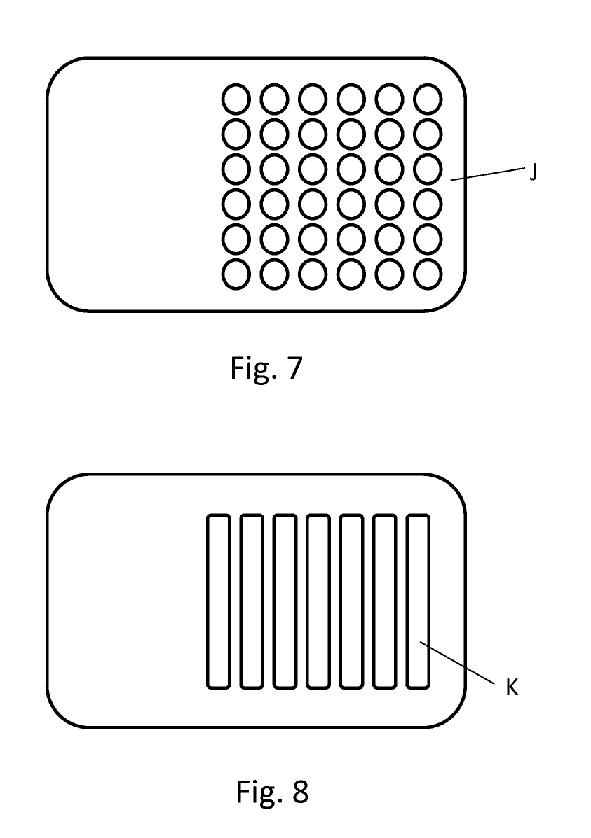

### DETAILED DESCRIPTION OF THE INVENTION

The preferred embodiment for the present invention presents a card style applicator that is coated (on one or both sides) with a deodorant or other cosmetic product. This card can be roughly the size of a credit card, with variations in height, width and thickness as required. Materials used for the card may be PVC or other plastics, papers, metals or known compounds with sufficient rigidity to allow a slight bending upon pressing on the human body while not degrading when exposed to the applied deodorant (or other) product.

A protective covering is included over the deodorant product. This cover is removed when the product is to be used. The cover can be removed through a ‘bend and break’ scoring that is scribed along 1 edge, or a tear strip that separates the cover along one of its edges, or a peel back action that causes the cover to no longer be attached to the card.

The deodorant or other cosmetic product can be applied to the card on one or both sides of the card, and in either a continuous, roughly rectangular patch, or a series of spaced apart dots, or a series of ribs, or any other pattern suitable for application and transfer to the body.

There is a space provided on the card that is free of deodorant or other cosmetic product so an individual can grasp it during use.

I claim:

1. A single use, disposable personal care device and method that consists of:
a flat card shaped device,
said flat card shaped device being constructed of a material that is semi-rigid and non-porous,
said flat card shaped device having 2 coplanar surfaces separated by a small distance in comparison to its width and length,
said coplanar surfaces being coated with a personal care product on either or both of said coplanar surfaces,
said personal care product being protected by a removable covering, wherein said single use, disposable device is used to apply said personal care product directly onto the human body, and subsequently disposed of after a single use.

2. The device and method of claim 1 wherein said width and length are similar to those of a credit card

3. The device and method of claim 1 wherein said personal care product covers substantially the entire surface area of said coplanar surfaces

4. The device and method of claim 1 wherein said personal care product covers a solid rectangular shaped area that is less than the entire surface area of said coplanar surfaces

5. The device and method of claim 1 wherein said personal care product covers a pattern of spaced apart circular shaped areas on said coplanar surfaces

6. The device and method of claim 1 wherein said personal care product covers a series of spaced apart bar shaped areas on said coplanar surfaces

7. The device and method of claim 1 wherein said coplanar surfaces have an area that is free of said personal care product to allow a user space to grasp and hold said flat card shaped device

8. The device and method of claim 1 wherein said removable covering is constructed of a material and design that causes the deodorant to be completely sealed and leak proof

9. The device and method of claim 1 wherein said removable covering is adhesively attached to each said coplanar surface and contains a graspable lift tab by which it can be separated from said coplanar surface

10. The device and method of claim 1 wherein said removable covering consists of 2 thin coplanar surfaces that are identical in shape to each other and slightly larger than said flat card shaped device, with said coplanar surfaces being connected to each other along all sides to effectively seal and contain said flat card shaped device within itself, said removable covering containing a scored bend/break line scribed across one end, said scored bend/break line creating an opening in said removable covering by which said flat card shaped device can be removed from said removable covering following the bending and breaking of said scored bend/break line

11. The device and method of claim 1 wherein said removable covering consists of 2 thin coplanar surfaces that are identical in shape to each other and slightly larger than said flat card shaped device, with said coplanar surfaces being connected to each other along all sides to effectively seal and contain said flat card shaped device within itself, said removable covering containing a tear strip across one end, said tear strip creating an opening in said removable covering by which said flat card shaped device can be removed from said removable covering following the removal of said tear strip.

12. The device and method of claim 1 wherein said personal care product is in either a solid or semi-solid gel state

13. The device and method of claim 1 wherein said personal care product is a deodorant product

14. The device and method of claim 1 wherein said personal care product is a cosmetic or cologne product

15. The device and method of claim 1 wherein said personal care product is completely used up following use

16. The device and method of claim 1 wherein said personal care product is not completely used up following use
